(19) United States
(12) Patent Application Publication
(10) Pub. No.: US 2002/0119939 A1
(43) Pub. Date: Aug. 29, 2002

(54) Stable aqueous deoxyfructosazine solution

(76) Inventors: Richard Bastin, Wiltshire (GB); Malcolm Charles Hart, Essex (GB); Nicholas Hughes, Essex (GB)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 09/754,175

(22) Filed: Jan. 4, 2001

Related U.S. Application Data
(60) Provisional application No. 60/188,350, filed on Mar. 9, 2000.

(30) Foreign Application Priority Data
Jan. 6, 2000 (FR) FR00/00138

(51) Int. Cl. A61K 31/4015

(52) U.S. Cl. 514/42; 514/424

(57) ABSTRACT

The present invention relates to a stable aqueous deoxyfructosazine solution.

### CROSS REFERENCE TO RELATED APPLICATIONS

This application claims the benefit of U.S. Provisional Application NO. 60/188,350, filed on Mar. 9, 2000, and of French Patent Application 00/00138 filed on Jan. 6, 2000.

### FIELD OF THE INVENTION

The present invention relates to a stable aqueous deoxyfructosazine solution, in particular for its pharmaceutical use for oral administration.

### BACKGROUND OF THE INVENTION

Deoxyfructosazine, or 2-((1R, 2S, 3R)1, 2, 3, 4-tetrahydroxybutyl)-5-((2′S, 3′R)2′, 3′, 4′-trihydroxy-butylpyrazine), is known for its antidiabetic properties (WO 97/28813).

The aqueous solutions thereof, and in particular those for oral administration, must be physicochemically stable for several weeks or months, and under variable temperature conditions.

### SUMMARY OF THE INVENTION

It has now been found that, in order to have maximum stability, aqueous deoxyfructosazine solutions should have a pH from 3 to 5, preferably from 3.5 to 4.5 and more particularly of 4.

Outside this pH range, the instability of the solution increases rapidly and the solution contains degradation products in an amount which is incompatible with pharmacological use. Thus, the percentage of a degradation product A becomes high and unacceptable when the pH of the aqueous solution decreases and the percentage of a degradation product B becomes high and unacceptable when the pH of the aqueous solution increases.

### DETAILED DESCRIPTION OF THE INVENTION

The pH of the aqueous solution is obtained by means of a pharmaceutically acceptable acid or a pharmaceutically acceptable buffer system comprising an acid and a base. Pharmaceutically acceptable acids which can be used, for example, are citric acid, phosphoric acid, acetic acid and tartaric acid. Bases which can be used in particular for the buffer system include trisodium citrate, disodium phosphate, disodium tartrate, sodium acetate and sodium hydroxide.

Preferably, the aqueous deoxyfructosazine solution is brought to a pH from 3 to 5, more preferably from 3.5 to 4.5 and still more preferably of 4, by means of a buffer system. Preferred buffer systems which may be mentioned are citric acid and trisodium citrate systems or phosphoric acid and sodium hydroxide systems.

The amount of deoxyfructosazine in the oral solution is from 1 to 150 mg/ml and in particular from 10 to 100 mg/ml.

The study of the stability of aqueous deoxyfructosazine solutions was carried out according to the following protocol:

A deoxyfructosazine solution (1 mg/ml) is prepared using an aqueous 0.05M phosphoric acid solution. Samples of the solution are adjusted to pH 2, 3, 4, 5, 6, 7, 8 and 9 by addition of sodium hydroxide. The solutions thus obtained are stored in glass bottles protected from the light, at 4° C. (control solution), 25° C., 45° C. and 60° C. After 2, 7 and 15 days, the samples are diluted 5-fold with a 20 mM, pH 7 phosphate buffer and the stability is examined by high pressure liquid chromatography (HPLC).

The results after storing the solutions for 15 days are as follows:

| | | |
|---|---|---|
| | | % of decomposition product |
| | | calculated relative to the |
| Storage | pH of the | total area of the peaks |

| temperature | solution | Product B | Product A |
|---|---|---|---|
| 45° C. | 2 | ND | 1.52 |
| | 3 | ND | 0.16 |
| | 4 | ND | ND |
| | 5 | 0.03 | ND |
| | 6 | 0.21 | ND |
| | 7 | 0.69 | ND |
| | 8 | 0.95 | ND |
| | 9 | 1.92 | ND |
| 60° C. | 2 | 0.02 | 6.94 |
| | 3 | 0.03 | 0.87 |
| | 4 | 0.05 | 0.07 |
| | 5 | 0.24 | ND |
| | 6 | 0.71 | ND |
| | 7 | 13.9 | ND |
| | 8 | 37.9 | ND |
| | 9 | 37.7 | ND |

ND = not detected

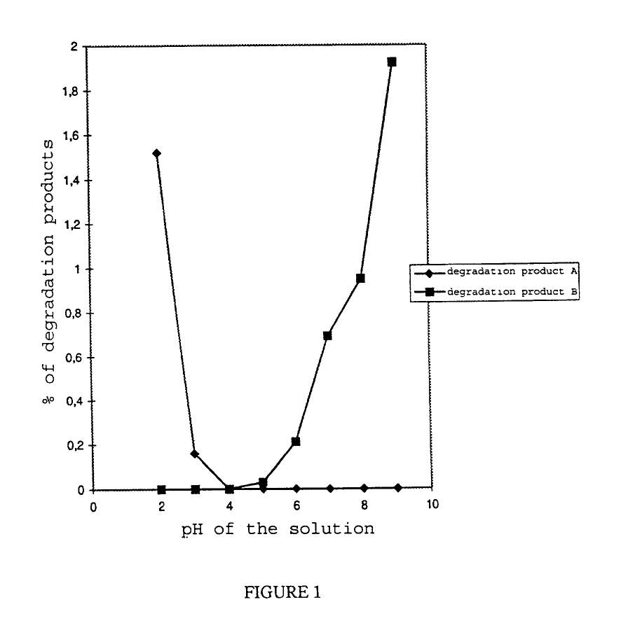

The result of the tests are also summarized in FIGS. 1 to 7.

FIG. 1 shows the percentages of the degradation products A and B formed at different pHs after storing the aqueous solutions for 15 days at 450° C.

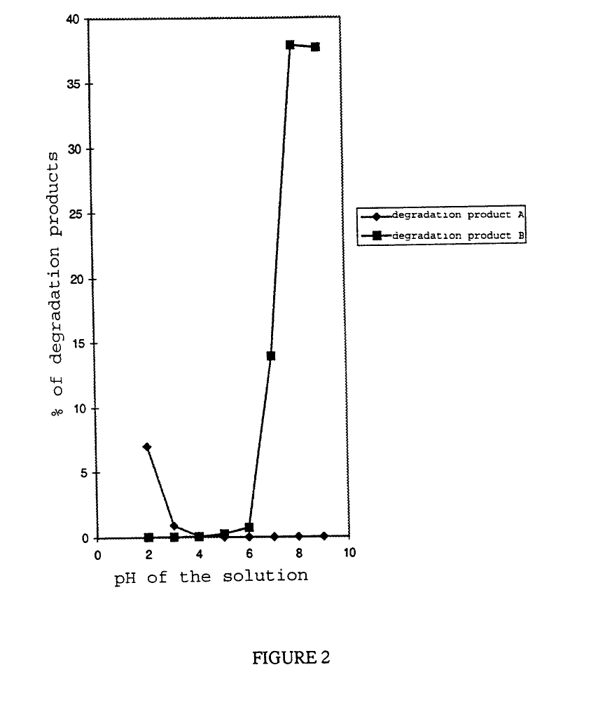

FIG. 2 shows the percentages of the degradation products A and B formed at different pHs after storing the aqueous solutions for 15 days at 60° C.

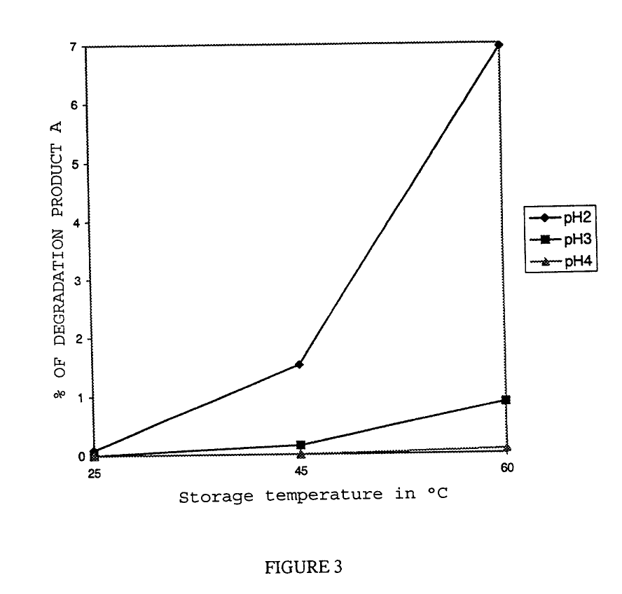

FIG. 3 shows the variation in the formation of the degradation product A after storing aqueous solutions at pH 2, 3 and 4 for 15 days at 25° C., 45° C. and 60° C.

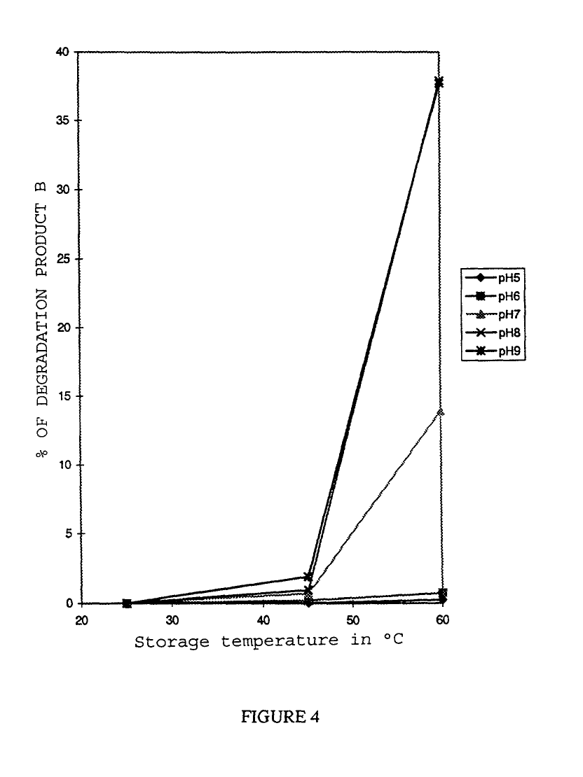

FIG. 4 shows the variation in the formation of the degradation product B after storing aqueous solutions at pH 2, 3 and 4 for 15 days at 25° C., 45° C. and 60° C.

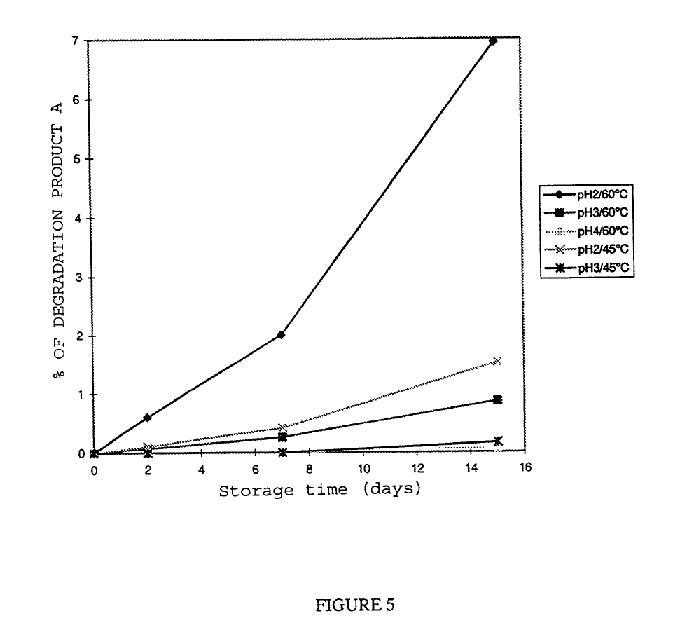

FIG. 5 shows the variation in the formation of the degradation product A as a function of the storage time of the aqueous solutions at pH 2 to 4 of solutions at 45 and 60° C.

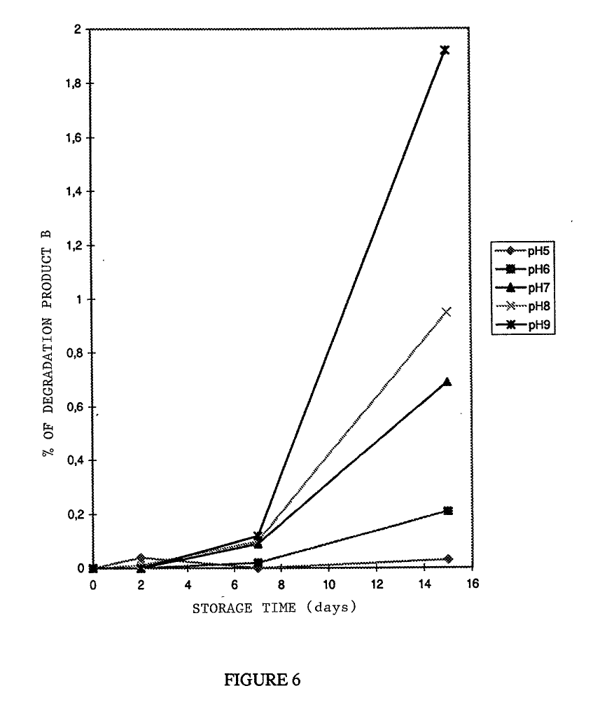

FIG. 6 shows the variation in the formation of the degradation product B as a function of the storage time of the aqueous solutions at pH 5 to 9 of solutions at 45° C.

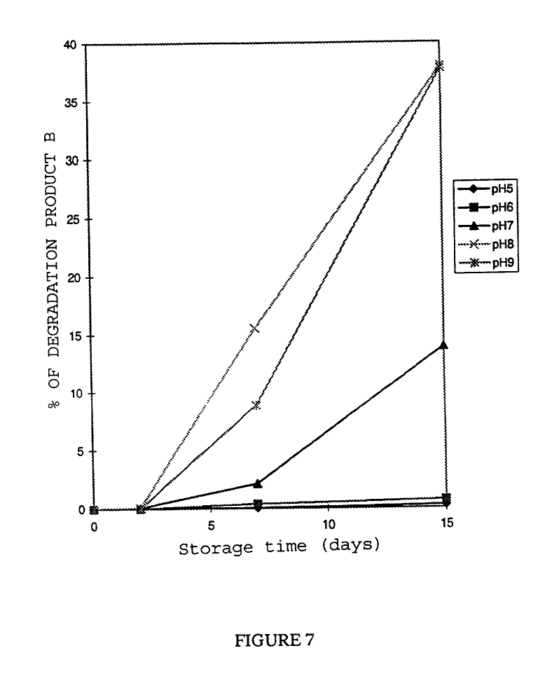

FIG. 7 shows the variation in the formation of the degradation product B as a function of the storage time of the aqueous solutions at pH 5 to 9 of solutions at 60° C.

The solutions for oral administration can contain buffers such as those mentioned above, diluents, for example ethanol, propylene glycol and glycerol, sweeteners such as sodium saccharin, cyclamate, thickeners such as sucrose, cellulose derivatives, Lycasin® and sorbitol, water-soluble flavorings, antimicrobial preserving agents such as sodium benzoate, benzoic acid and colorants. They can optionally be sterilized by any method known to those skilled in the art which prevents the solution from being degraded.

The example below of an aqueous oral solution illustrates an embodiment of the invention:

100 mg of deoxyfructosazine

0.5 ml of propylene glycol

70 mg of citric acid monohydrate

50 mg of trisodium citrate dihydrate

20 mg of sodium benzoate

5 mg of sodium saccharin

2 μl of pineapple flavoring

0.8 mg of caramel

0.1 mg of riboflavin

demineralized water in a quantity sufficient for 10 ml of solution.

The solutions according to the invention can be prepared by dissolving, with stirring, at a temperature in the region of 20° C., deoxyfructosazine and the optional excipients in distilled water and adjusting the pH using a pharmaceutically acceptable acid. When a buffer system is used, the deoxy-fructosazine and the optional excipients can also be dissolved in distilled water containing a pharmaceutically acceptable acid, and the pH can be adjusted by means of the base in the buffer system.

These solutions are useful for preventing and/or treating type 2 diabetes and its complications.

1. An aqueous deoxyfructosazine solution having a pH from 3 to 5.

2. A solution according to claim 1, having a pH from 3.5 to 4.5.

3. A solution according to claim 2, having a pH of 4.

4. A solution according to claim 1, containing 1 to 150 mg/ml of deoxyfructosazine.

5. A solution according to claim 4, containing 10 to 100 mg/ml of deoxyfructosazine.

6. A solution according to claim 1, wherein the pH is achieved by means of a pharmaceutically acceptable acid.

7. A solution according to claim 6, wherein the pharmaceutically acceptable acid is selected from citric acid, phosphoric acid, tartaric acid and acetic acid.

8. A solution according to claim 1, wherein the pH is achieved by means of a pharmaceutically acceptable buffer system.

9. A solution according to claim 8, wherein the buffer system comprises a pharmaceutically acceptable acid and a pharmaceutically acceptable base.

10. A solution according to claim 9, wherein the acid in the buffer system is selected from citric acid, phosphoric acid, tartaric acid and acetic acid.

11. A solution according to claim 9, wherein the base in the buffer system is selected from trisodium citrate, disodium phosphate, disodium tartrate and sodium hydroxide.

12. A solution according to claim 8, wherein the buffer system comprises citric acid and sodium citrate.

13. A solution according to claim 8, wherein the buffer system comprises phosphoric acid and sodium hydroxide.

14. A solution according to claim 1, which is suitable for oral administration.

15. A solution according to claim 1, which contains one or more pharmaceutically acceptable diluents.

16. A pharmaceutical composition containing a solution according to claim 1.

17. A pharmaceutical composition according to claim 16, which is suitable for oral administration.

18. A pharmaceutical composition according to claim 17, comprising 100 mg of deoxyfructosazine, 0.5 ml of propylene glycol, 70 mg of citric acid monohydrate, 50 mg of trisodium citrate dihydrate, 20 mg of sodium benzoate, 5 mg of sodium saccharin, 2 μl of pineapple flavoring, 0.8 mg of caramel, 0.1 mg of riboflavine, and demineralized water in a quantity sufficient for 10 ml of solution.
